(19) United States
(12) Patent Application Publication
(10) Pub. No.: US 2011/0034892 A1
(43) Pub. Date: Feb. 10, 2011

(54) Passive wound therapy device

(76) Inventor: John Buan, Maple Grove, MN (US)

Correspondence Address: FACTOR & LAKE, LTD, 1327 W. WASHINGTON BLVD., SUITE 5G/H, CHICAGO, IL, 60607, US

(21) Appl. No.: 12/462,846

(22) Filed: Aug. 10, 2009

(51) Int. Cl. A61F 13/02; A61F 13/15

(52) U.S. Cl. 604/367; 604/385.03

(57) ABSTRACT

A wound therapy device which may have a first configuration to passively absorb exudates from a wound and a second configuration to absorb exudates from the wound while the device is subjected to a negative pressure. An absorptive pad may be configured to passively absorb the removed exudates.

### RELATED APPLICATIONS

None.

### TECHNICAL FIELD

The present invention relates, in general, to a device and method for wound therapy that is capable of treating a variety of chronic and acute wound types, including, but not limited to, infection wounds, venous ulcers, arterial ulcers, diabetic ulcers, burn wounds, post amputation wounds, surgical wounds, and the like.

### BACKGROUND

Negative pressure therapy has been one method used for the treatment of a variety of wounds by practitioners in the art. Conventional negative pressure therapy devices are generally large in size and often require the use of complicated equipment such as suction pumps, vacuum pumps and complex electronic controllers.

However, it is believed that in some instances, a wound device that is capable of passively absorbing fluid and has the ability to be used in with negative pressure wound therapy would be beneficial. A clinician could place the device and allow it to passively absorb fluids and exudates, and, at a later time, without changing the device or otherwise exposing the wound to air, administer negative pressure wound therapy to the wound to facilitate healing.

### BRIEF SUMMARY OF THE INVENTION

In one embodiment of the invention, the invention provides a wound therapy device comprising a backing material having a shape with an edge and a first side having an adhesive and a second side, a port hole disposed in the backing material, and, an absorptive pad disposed on the first side of the backing material such that a portion of the absorptive pad is disposed under the port hole, wherein the absorptive pad is comprised of a material that passively draws exudates from a wound and is capable of retaining the exudates while the device is subjected to negative pressure.

In another embodiment of the invention, the invention provides a device with a gasket disposed on the backing material distally between the absorptive pad and the edge.

In a further embodiment of the invention, the invention provides a device with the absorptive pad having a shape and the gasket having a shape, and wherein the shape of the absorptive pad is substantially the same as the shape of the gasket.

In still another embodiment of the invention, the invention provides a device with the shape of the absorptive pad being an oval.

In yet another embodiment of the invention, the invention provides a device with the gasket being a hydrogel and having a width of approximately ⅜ of an inch.

In a further embodiment of the invention, the invention provides a device with the gasket having a thickness of approximately 30 mils.

In another embodiment of the invention, the invention provides a device with the gasket being disposed immediately adjacent the absorptive pad.

In still another embodiment of the invention, the invention provides a device with a wound interface layer disposed around an exposed portion of the absorptive pad.

In a further embodiment of the invention, the invention provides a device with the wound interface layer being a silver plated mesh.

In a different embodiment of the invention, the invention provides a device with the backing material being semi-permeable.

In yet another embodiment of the invention, the invention provides a device with the port hole further including a fluid impermeable membrane.

In still another embodiment of the invention, the invention provides a device with a viewing portal disposed in the backing material proximate the port hole.

In another embodiment of the invention, the invention provides a device with a liner disposed on at least a portion of the adhesive.

In an embodiment of the invention, the invention provides a device with a second liner disposed on a portion of the edge.

In a further embodiment of the invention, the invention provides a device with a third liner disposed on a second portion of the edge.

In still another embodiment of the invention, the invention provides a device with a backing material having a shape with an edge and a first side having an adhesive and a second side, and, an absorptive pad disposed on the first side of the backing material, wherein the absorptive pad is comprised of a material that passively draws exudates from a wound and is capable of retaining the exudates while the device is subjected to a negative pressure, and, wherein the device is capable of being used with the negative pressure.

In yet another embodiment of the invention, the invention provides a device that includes a backing material, and, an absorptive pad disposed adjacent the backing material, wherein the device has a first configuration to passively absorb exudates from a wound and the device has a second configuration to absorb exudates from the wound while the device is subjected to a negative pressure.

### BRIEF DESCRIPTION OF THE DRAWINGS

The present invention will become more fully apparent from the following description and appended claims, taken in conjunction with the accompanying drawings. Understanding that the accompanying drawings depict only typical embodiments, and are, therefore, not to be considered to be limiting of the scope of the present disclosure, the embodiments will be described and explained with specificity and detail in reference to the accompanying drawings as provided below.

FIG. 1 is a cutaway side view of an embodiment of a wound therapy device according to the present invention.

FIG. 2 is a top view of an embodiment of a wound therapy device according to the present invention.

FIG. 3 is an exploded view of an embodiment of a wound therapy device according to the present invention.

FIG. 4 is a back view of an embodiment of a wound therapy device according to the present invention.

### DETAILED DESCRIPTION OF THE DRAWINGS

It will be readily understood that the components of the embodiments as generally described and illustrated in the Figures herein could be arranged and designed in a wide variety of different configurations. Thus, the following more detailed description of various embodiments, as represented in the Figures, is not intended to limit the scope of the present disclosure, but is merely representative of various embodiments. While the various aspects of the embodiments are presented in drawings, the drawings are not necessarily drawn to scale unless specifically indicated.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

In the following description, numerous specific details are provided to provide a thorough understanding of embodiments of the invention. One skilled in the relevant art will recognize, however, that the invention can be practiced without one or more of the specific details, or with other methods, components, materials, and so forth. In other instances, well-known structures, materials, or operations such as vacuum sources are not shown or described in detail to avoid obscuring aspects of the invention.

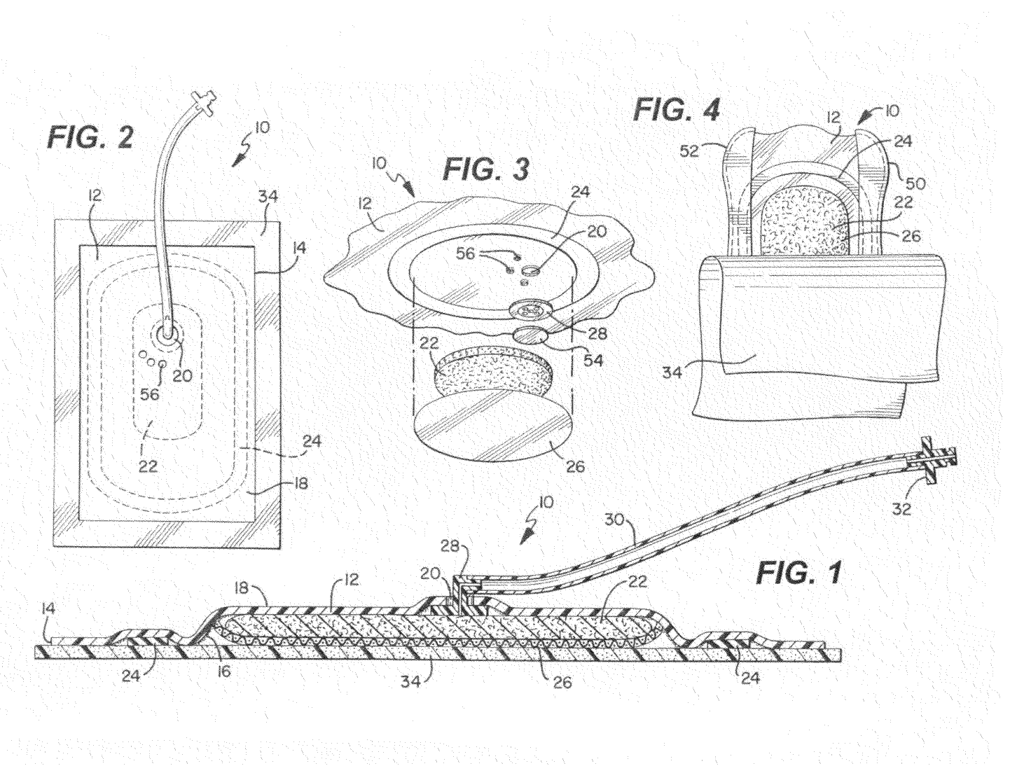

Referring now to FIGS. 1-4, a wound therapy device 10 is shown. A wound therapy device 10 includes a backing material 12 having a shape with an edge 14 and a first side 16 having an adhesive and a second side 18. The present invention contemplates multiple shapes including, but not limited to, circles, ovals, squares, and oblongs. The backing material 12 may be flexible to allow the device 10 to be contoured to the appropriate location of a wound. In addition, it is preferred that the backing material 12 be semi-permeable. What is meant by the term semi-permeable is that the backing material has breathability aspects that do not impact the ability to hold negative pressure relative to appropriate therapeutic treatment, as would be understood by those of ordinary skill in the art.

An absorptive pad 22 is disposed adjacent the first side 16 of the backing material 12. The absorptive pad 22 is capable of passively absorbing exudates and liquid from a wound. Specifically, it is meant by the term “passively” that the absorptive pad 22 acts to absorb liquids from the wound without the use of negative pressure. For example, the absorptive pad 22 may use capillary action to draw exudates/liquid out of the wound. However, since the device 10 may be, but is not necessarily, utilized with negative pressure, the pad 22 should also preferably be capable of continuing to hold the exudates/liquid while allowing the device to communicate negative pressure to the wound. The absorptive pad 22 may be a material such as sponges, foams, fibers, wicking fibers, hollow fibers, beads, fabrics, or gauzes, super-absorbent materials including super-absorbent polymers in various forms, absorbent foams, gelling agents such as sodium carboxy methyl cellulose, packing materials, and/or combinations thereof.

A port hole 20 may be disposed in the backing material 18. Since the present wound therapy device 10 is used in a passive manner, the port hole 20 would be included, only to make the device 10 capable of operating with a negative pressure method, as well as passively. The port hole 20 can have any shape and size. In an embodiment of the present invention, an adaptor 28 is disposed within the port hole 20. In a preferred embodiment of the invention, a fluid impermeable membrane 54 is disposed on the adaptor 28. The fluid impermeable membrane 54 prohibits fluids and other exudates from flowing from the device 10 to the source of the negative pressure. In a preferred embodiment, the fluid impermeable membrane 54 is GORE-TEX®; however, other materials are contemplated to be used.

The absorptive pad 22 may be disposed under the port hole 20. By used of the term “under,” it is meant that when the device 10 is viewed from a top view (such as in FIG. 2), a portion of the absorptive pad 22 is disposed beneath the port hole 20. In addition, it is contemplated that the absorptive pad 22 need not be directly beneath or under the port hole 20, i.e., other structures may be disposed between the two structures.

It is contemplated that a liner 34 is removably attached to a portion of the first side 16 of the backing material 12. In a preferred embodiment, a second liner 50 and a third liner 52 each disposed on a portion of the edge 14. This is to facilitate quick deploy and use of the device 10. For example, the liner 34 can be removed. The clinician or patient placing the device 10 can utilize second liner 50 and third liner 52 while placing the device 10 without touching the adhesive on the first side 16.

In order to facilitate the device 10 being used for a negative pressure therapy, a tube segment 30 may be utilized to allow for communication between the adaptor 28 and a connector 32. The connector 32 is connected (either directly or indirectly) to the source of the negative pressure (not shown). The tube segment 30 is long enough that the connector 32 is distally spaced from the adaptor 28 and/or port hole 20. Non-kinking tube material may be used. It is contemplated that the adaptor 28, tube segment 30 and connector 32 is comprised of one structure or multiple structures connected. By moving the connector 32 away from the port hole 20, it is believed that the device will increase the comfort of the patient, since the connection between the device and pump does not have to be located under gauze or other wrapping material such as Unna Boot or COBAN® which typically wraps around the device. Additionally, if the connection between the pump and device needs to be broken, the wrapping material does not need to be removed.

It is contemplated that the device 10 includes at least one viewing portal 56 in the backing material 12. Since the absorptive pad 22 retains the exudates and fluids removed from the wound, the viewing portal 56 allows a clinician or patient to determine if the absorptive pad 22 is saturated or not. It is preferred that the viewing portal 56 be disposed between the wound and the port hole 20, should the device 10 include a port hole 20. In addition, since certain backing materials are non-transparent it is contemplated that a semi-transparent material be disposed over the viewing portal 56 so as to prevent any exudates from leaking out.

It is contemplated that the device 10 also includes wound interface layer 26, or other similar structure, disposed around a portion of the absorptive pad 22. The wound interface layer 26 will allow epithelialization and reduce wound tissue adherence to the device. In one embodiment, the wound interface layer 26 may comprise a silver plated mesh, such as one that is currently commonly available and known as SILVERION®.

A gasket 24 is disposed on the backing material 12, and more particularly on the first side 16 of the backing material 12 with the adhesive. The gasket 24 is disposed distally between the edge 14 of the backing material 12 and the absorptive pad 22. It is contemplated that the gasket 24 be disposed immediately adjacent to the absorptive pad 22.

The gasket 24 has a thickness, and it is contemplated that the thickness of the gasket 24 is between 3 to 5 mils and the width of the gasket 24 is approximately ⅜ of an inch.

In one embodiment of the invention the gasket 24 is a hydrogel. Such materials are currently available from Katecho, in Des Moines, Iowa (USA). It is preferred that the gasket 24 be a material that be biocompatible with skin. In addition the gasket 24 material should mildly adhere to the skin, but not adhere to the skin in the same manner as the adhesive on the backing material. In addition, the gasket 24 material should be mildly flowable. Furthermore, the gasket 24 material should be non-reactive to normal medical device sterility processes. Another contemplated material is a silicone gel; however, it is currently believed to be too cost prohibitive to utilize the silicone gel.

It is preferred that the gasket 24 and the absorptive pad 22 have the same (relatively) shape. It is most preferred that the shape is an oval. Moreover, it is important that the gasket 24 be sized such that the wound is entirely disposed within the gasket 24.

Such a gasket 24 is believed to minimize the possibility of air pressure leaks, and thus increase the efficiency of the device, especially when used in a negative pressure wound therapy.

Without further elaboration, it is believed that one skilled in the art can use the preceding description to utilize the present disclosure to its fullest extent. The examples and embodiments disclosed herein are to be construed as merely illustrative and not a limitation of the scope of the present disclosure in any way. It will be apparent to those having skill in the art that changes may be made to the details of the above-described embodiments without departing from the underlying principles of the disclosure provided herein. In other words, various modifications and improvements of the embodiments specifically disclosed in the description above are within the scope of the appended claims. Note that elements recited in means-plus-function format are intended to be construed in accordance with 35 U.S.C. §112 ¶6. The scope of the invention is therefore defined by the following claims.

1. A wound therapy device comprising:
a backing material having a shape with an edge and a first side having an adhesive and a second side;
a port hole disposed in the backing material; and,
an absorptive pad disposed on the first side of the backing material such that a portion of the absorptive pad is disposed under the port hole, wherein the absorptive pad is comprised of a material that passively draws exudates from a wound and is capable of retaining the exudates while the device is subjected to negative pressure.

2. The device of claim 1 further comprising a gasket disposed on the backing material distally between the absorptive pad and the edge.

3. The device of claim 2 further comprising the absorptive pad having a shape and the gasket having a shape, and wherein the shape of the absorptive pad is substantially the same as the shape of the gasket.

4. The device of claim 3 further comprising the shape of the absorptive pad being an oval.

5. The device of claim 2 further comprising the gasket being a hydrogel and having a width of approximately ⅜ of an inch.

6. The device of claim 5 further comprising the gasket having a thickness of approximately 30 mils.

7. The device of claim 2 further comprising the gasket being disposed immediately adjacent the absorptive pad.

8. The device of claim 1 further comprising a wound interface layer disposed around an exposed portion of the absorptive pad.

9. The device of claim 8 wherein the wound interface layer is a silver plated mesh.

10. The device of claim 1 wherein the backing material is semi-permeable.

11. The device of claim 1 wherein the port hole further comprises a fluid impermeable membrane.

12. The device of claim 1 further comprising a viewing portal disposed in the backing material proximate the port hole.

13. The device of claim 1 further comprising a liner disposed on at least a portion of the adhesive.

14. The device of claim 13 further comprising a second liner disposed on a portion of the edge.

15. The device of claim 14 further comprising a third liner disposed on a second portion of the edge.

16. A wound therapy device comprising:
a backing material having a shape with an edge and a first side having an adhesive and a second side; and,
an absorptive pad disposed on the first side of the backing material, wherein the absorptive pad is comprised of a material that passively draws exudates from a wound and is capable of retaining the exudates while the device is subjected to a negative pressure, and,
wherein the device is capable of being used with the negative pressure.

17. A wound therapy device for removing exudates from a wound, the device comprising:
a backing material; and,
an absorptive pad disposed adjacent the backing material;
wherein the device has a first configuration to passively absorb exudates from a wound and the device has a second configuration to absorb exudates from the wound while the device is subjected to a negative pressure.
